(19) United States
(12) Patent Application Publication
(10) Pub. No.: US 2026/0045336 A1
(43) Pub. Date: Feb. 12, 2026

(54) HEALTH DATA MANAGEMENT DEVICE, HEALTH DATA MANAGEMENT PROGRAM, AND HEALTH DATA MANAGEMENT METHOD

(71) Applicant: FUJIFILM Corporation, Tokyo (JP)

(72) Inventor: Takanobu OTSUBO, Tokyo (JP)

(73) Assignee: FUJIFILM Corporation, Tokyo (JP)

(21) Appl. No.: 19/366,529

(22) Filed: Oct. 23, 2025

Related U.S. Application Data
(63) Continuation of application No. PCT/JP2024/016064, filed on Apr. 24, 2024.

(30) Foreign Application Priority Data
Apr. 27, 2023 (JP) 2023-073406

(51) Int. Cl. G16H 10/60

(52) U.S. Cl. CPC G16H 10/60

(57) ABSTRACT

A health data management device includes a processor, in which the processor is configured to acquire affiliation information of an output destination of a plurality of health data measured under different measurement conditions, intended-use information, an output condition, determine a reliability degree for the health data from the measurement conditions based on the affiliation information and the intended-use information, and control whether or not to output the health data depending on the reliability degree and the output condition.

### CROSS-REFERENCE TO RELATED APPLICATIONS

This application is a Continuation of PCT International Application No. PCT/JP2024/016064 filed on 24 Apr. 2024, which claims priority under 35 U.S.C § 119(a) to Japanese Patent Application No. 2023-073406 filed on 27 Apr. 2023. The above application is hereby expressly incorporated by reference, in its entirety, into the present application.

### BACKGROUND OF THE INVENTION

### 1. Field of the Invention

The present invention relates to a health data management device, a non-transitory computer readable medium storing a health data management program, and a health data management method.

### 2. Description of the Related Art

In a case where health information of a plurality of individuals managed at health checkup facilities, medical institutions, and on personal information terminals is utilized at another facility, the health information may be unusable by the facility that utilizes the information depending on a measurement condition of the health information.

WO2015/186631A discloses a method of displaying data such as blood pressure and pulse waves in a way that allows a user to distinguish at a glance whether the data is highly reliable or relatively less reliable by distinguishing between display methods based on whether the data is obtained from a certified medical device. In addition, it is disclosed that, in a case where the data is presented to an expert such as a doctor, a display method or a display mode is changed to clearly display whether the data is obtained using a highly reliable measurement device.

### SUMMARY OF THE INVENTION

On the other hand, WO2015/186631A discloses changing the display according to a status of an output destination based on whether the user is an expert such as a doctor, but does not disclose controlling output of health data depending on environment of the output destination, and the output health data may be unusable because the content of the output health data is not suitable for its intended purpose.

An object of the present invention is to provide a health data management device, a non-transitory computer readable medium storing a health data management program, and a health data management method that output health data with content suitable for its intended purpose.

A health data management device according to an aspect of the present invention comprises: a processor, in which the processor is configured to acquire affiliation information of an output destination of a plurality of health data measured under different measurement conditions and intended-use information of the health data, and control output of the health data for each measurement condition based on the affiliation information and the intended-use information.

It is preferable that the measurement conditions include at least one of the following items: a measurement date and time; a measurement device; a measurement method; a measurement location; and a qualification of a measurer, and the processor is configured to determine a reliability degree for the health data from the measurement conditions, and control the output of the health data according to the reliability degree.

It is preferable to perform weighting on the item included in the measurement conditions from the affiliation information and the intended-use information, and correct the reliability degree in accordance with the measurement conditions for which the weighting is performed.

It is preferable to perform the weighting to a greater extent in a case where the measurer is a qualified person who has a specific qualification than in a case where the measurer is an unqualified person.

It is preferable that the output destination sets a required reliability degree that is a lower limit value of the reliability degree of the health data whose output is received, and that the processor is configured to compare the required reliability degree with the reliability degree of the health data for each measurement condition, and determine an output condition for outputting the health data according to a comparison result.

It is preferable to output the health data without changing the output condition in a case where the reliability degree satisfies the required reliability degree, and output the health data by changing the output condition in a case where the reliability degree does not satisfy the required reliability degree.

It is preferable that the output condition is a fee paid by the output destination to an output source for the output of the health data, and that, in a case where the reliability degree does not satisfy the required reliability degree, the fee is changed to a lower price than a normal fee.

It is preferable to, in a case where there are the plurality of health data for the same examinee, output the health data having a highest reliability degree.

It is preferable to notify the output destination of the reliability degree of the health data to be output.

It is preferable to, in a case of outputting a health data group consisting of the plurality of health data, set a minimum value of the reliability degrees provided for the plurality of health data as a reliability degree of the health data group.

It is preferable to determine an output range of the health data for each measurement condition based on the affiliation information and the intended-use information, and output the health data according to the output range.

It is preferable to determine a case tendency value, which is a value indicating a tendency of a specific case, from a plurality of examination values included in the health data, and to, in a case where the intended-use information includes information on a required case tendency value, control the output of the health data based on the case tendency value corresponding to the intended-use information.

It is preferable to receive a search for the health data from the output destination and output the health data specified by the search.

A non-transitory computer readable medium storing a health data management program according to another aspect of the present invention causes a computer to execute: a function of acquiring affiliation information of an output destination of a plurality of health data measured under different measurement conditions and intended-use information of the health data; and a function of controlling output of the health data for each measurement condition based on the affiliation information and the intended-use information.

A health data management method according to still another aspect of the present invention comprises: a step of acquiring affiliation information of an output destination of a plurality of health data measured under different measurement conditions and intended-use information of the health data; and a step of controlling output of the health data for each measurement condition based on the affiliation information and the intended-use information.

According to the present invention, the health data to be output has a content suitable for its intended purpose.

### BRIEF DESCRIPTION OF THE DRAWINGS

FIG. 1 is a schematic diagram of a health data management system.

FIG. 2 is an explanatory diagram showing an example of devices connected to the health data management device.

FIG. 3 is a block diagram showing functions of the health data management device.

FIG. 4 is an explanatory diagram illustrating association of reliability degree data and case tendency data with health data.

FIG. 5 is an explanatory diagram illustrating generation of reliability degree data from measurement condition data.

FIG. 6 is an explanatory diagram illustrating generation of case tendency data from health data.

FIG. 7 is an explanatory diagram illustrating correction of reliability degree data using a weighting pattern of “subject recruitment” as an intended use.

FIG. 8 is an explanatory diagram illustrating correction of reliability degree data using a weighting pattern of “insurance plan proposal” as an intended use.

FIG. 9 is a flowchart showing a series of flows for controlling output of health data in which a reliability degree of a measurement condition is calculated based on output destination information.

FIG. 10 is a schematic diagram of a health data management system according to a second embodiment.

FIG. 11 is a block diagram showing functions of a health data utilization device according to the second embodiment.

FIG. 12 is an explanatory diagram of a list display of search results in the second embodiment.

FIG. 13 is an explanatory diagram of a list display of images of search results in the second embodiment.

### DESCRIPTION OF THE PREFERRED EMBODIMENTS

### First Embodiment

In the present invention, in managing a plurality of health data measured under different measurement conditions, a health data management device 11 acquires, from a health data utilization device 12, affiliation information of an output destination of the health data and intended-use information of the health data in the output destination, and controls output of the health data for each measurement condition using a method such as calculation of a reliability degree of the health data based on the affiliation information and the intended-use information.

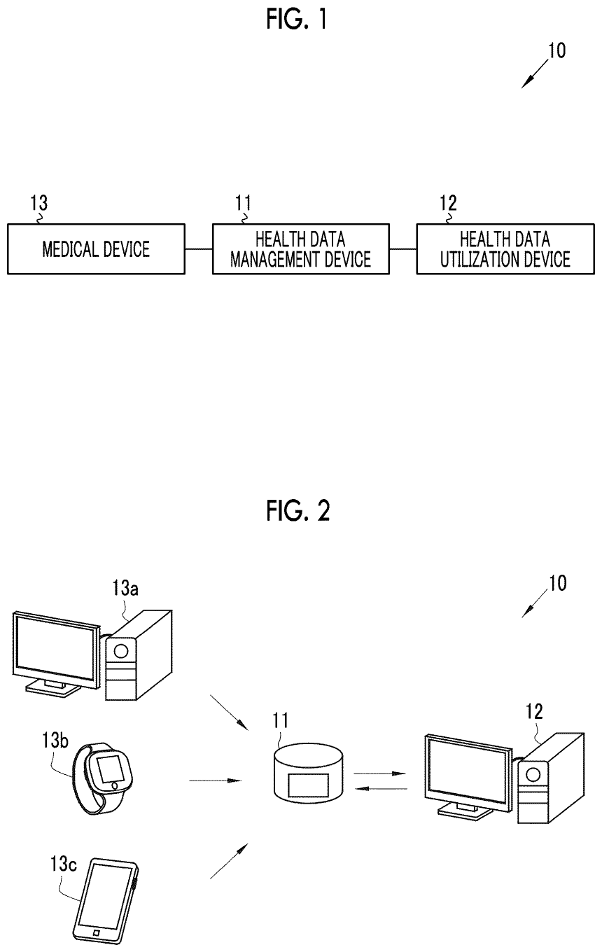

FIG. 1 is a diagram showing a configuration example of a health data management system 10 that implements a health data management method according to an embodiment of the present invention. The health data management system 10 includes the health data management device 11, the health data utilization device 12, and a medical device 13, and the health data management device 11 communicates with the health data utilization device 12 and the medical device 13. The health data management device 11 acquires output destination information including information on a user and an intended use from the health data utilization device 12 and health data from the medical device 13, and calculates a reliability degree of each of the plurality of health data using the output destination information.

In addition, the health data management device 11 may be electrically connected to a display (not shown) and a user interface (UI) (not shown). The display displays the acquired various data, a calculation result of the reliability degree, and the like. The user interface is an input device for the user to input settings to the health data management device 11, and includes a keyboard, a mouse, and the like.

The health data utilization device 12 is an output destination of the health data management device 11, such as a server or a device, owned by a medical institution or an insurance company, which uses the health data for any intended use, and transmits, to the health data management device 11, affiliation information, intended-use information indicating the intended use of the health data, and information on the required health data as the output destination information.

The medical device 13 is a measurement device that measures the health data or a server that manages the health data acquired from the measurement device or user input, and performs distributed management of the health data or transmits the health data to the health data management device 11.

The health data is examination results obtained by measuring health-related items using respective measurement methods, for example, results obtained by acquiring data related to blood pressure, heart rate, electrocardiogram, brain wave, body fat, body temperature, blood, and the like of each examinee through each examination, and the blood data includes oxygen saturation, blood glucose level, hemoglobin level, and the like. In addition, the health data may include medical images such as a computed tomography (CT) image, a magnetic resonance imaging (MRI) image, an X-ray image, and an endoscopic image. The health data records measurement conditions at the time of measurement for each examination.

As shown in FIG. 2, in the health data management system 10, the health data management device 11 that outputs the health data to the health data utilization device 12 based on the output destination information can be connected to a plurality of types of domestic and overseas devices as the medical device 13, for example, medical devices 13a, 13b, and 13c, and can acquire the health data managed in a distributed manner. All of the medical devices 13a to 13c are devices that measure or manage health data, the medical device 13a is a server that manages health data, the medical device 13b is a wearable device, and the medical device 13c is an information terminal owned by an individual.

The medical device 13a is a server that is provided in each medical institution or medical-related company and that manages health data, and receives input of health data from a medical worker or a measurement device. The medical device 13b is a wearable device such as a smart watch, which is owned by a hospital or a health checkup center and loaned to the examinee in a case of conducting a health checkup to measure health data, or owned by an individual to measure and record health data on a daily basis. The medical device 13c is a personal information terminal such as a smartphone, and manages health data measured by a home medical device such as a health meter, a blood pressure meter, and a thermometer.

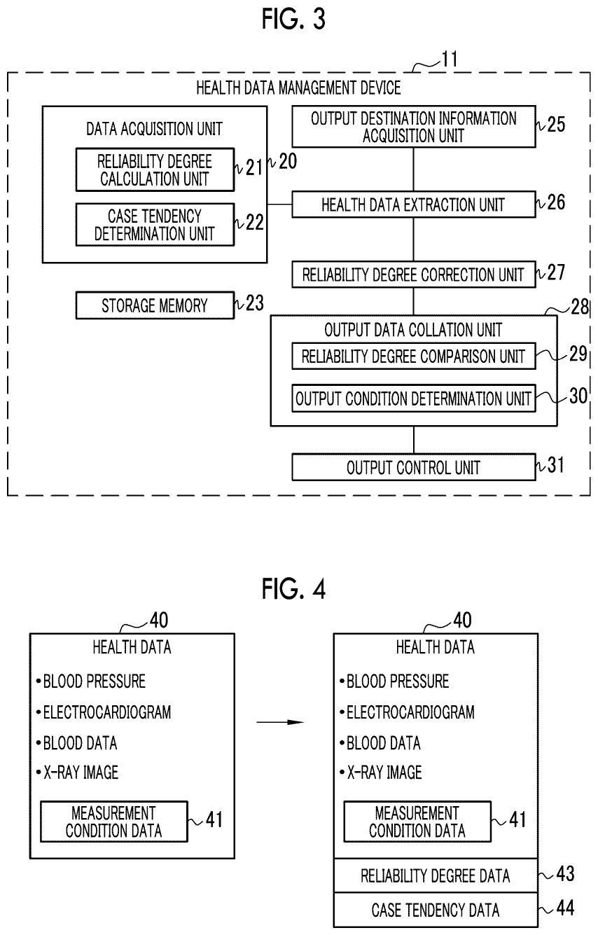

As shown in FIG. 3, in the health data management device 11, a program in a program memory is operated by a central control unit (not shown) constituted by a data control processor, so that functions of a data acquisition unit 20, an output destination information acquisition unit 25, a health data extraction unit 26, a reliability degree correction unit 27, an output data collation unit 28, an output control unit 31, and an input reception unit (not shown) are realized, functions of a reliability degree calculation unit 21 and a case tendency determination unit 22 are realized with the realization of the function of the data acquisition unit 20, and functions of a reliability degree comparison unit 29 and an output condition determination unit 30 are realized with the realization of the function of the output data collation unit 28. The input reception unit is connected to a user interface and receives an operation from the user.

The data acquisition unit 20 acquires health data having measurement conditions from the medical device 13 to which the health data management device 11 is connected. The acquired health data is used to calculate a reliability degree and determine a case tendency. Since the health data may include personal information of the examinee, it is preferable to acquire the health data using a dedicated line.

As shown in FIG. 4, health data 40 acquired by the data acquisition unit 20 includes at least measurement condition data 41 and an examination result of each examination, the reliability degree calculation unit 21 acquires reliability degree data 43 from the measurement condition data 41, and the case tendency determination unit 22 acquires case tendency data 44 from each examination result. The acquired reliability degree data 43 and case tendency data 44 are automatically added to the health data 40. In a case where the reliability degree correction is not performed in real time, the health data 40 associated with the reliability degree data 43 and the case tendency data 44 is preferably transmitted to a storage memory 23 and stored therein.

The reliability degree calculation unit 21 evaluates a reliability degree of each item included in the measurement condition data 41. The measurement conditions included in the measurement condition data 41 include at least one of a measurement device, a measurement method, a measurement location, a qualification of a measurer, or a measurement date and time. In addition, an overall reliability degree, which is an overall reliability degree of the measurement conditions, is calculated from a ratio of a total value of the reliability degrees of the items to a total value of the maximum attainable reliability degrees of the items of the measurement conditions. In addition, it is preferable that a representative value of the overall reliability degree for each measurement condition is used as a reliability degree of the health data 40. That is, the reliability degree data 43 for the health data 40 is determined from each measurement condition, and the output of the health data 40 is controlled according to the reliability degree.

The reliability degree of each item is calculated by, for example, evaluating the reliability degree with a three-stage score from 0 to 2 based on a reliability degree calculation table (not shown) stored in advance. Since the measurement condition data 41 is provided for each performed examination, the health data 40 having the examination results of a plurality of examinations has a plurality of reliability degree data 43.

A reliability degree of the measurement device is determined based on performance such as accuracy and precision of a measurement result output by the measurement device. For example, the higher the performance of the measurement device, the higher the reliability degree is set, whereas a measurement device having only minimum performance is assigned a lower reliability degree. In a case where the measurement device is unknown or an appropriate measurement device is not used, the reliability degree is set to the minimum value. In addition, the age of the measurement device may be included in the evaluation of the reliability degree.

A reliability degree of the measurement method is an item for evaluating whether the measurement method allows for traceability of the examination result such as a medical image or an examination value, or whether the measurement method is prone to errors or measurement mistakes in the examination result. For example, a method that requires visual confirmation of a scale on the measurement device, and in which examination values are likely to vary depending on the measurer, or a method that is not commonly used is rated lower.

A reliability degree of the measurement institution is determined using the past records of measurement and provision of the health data 40 for the institution that has performed the medical examination.

A reliability degree of the measurer qualification is an item evaluated based on whether the measurer in the examination is a qualified person who has a specific qualification such as a doctor or a nurse. It is preferable that the reliability degree is rated at the highest value in a case of being the qualified person and is rated at the minimum value in a case of not being the qualified person. The reliability degree may be finely classified according to the type of qualification of the measurer.

A reliability degree of the doctor's audit is an item in which the presence or absence of the implementation of the doctor's audit is reflected as the reliability degree on the health data 40. For example, in a case where there is a doctor's audit, the reliability degree is rated at the highest value, and, in a case where there is no audit, the reliability degree is rated at the minimum value. The audit involves checking records of examination results such as each examination value and medical images, measurement conditions, and the like.

A reliability degree of the measurement date and time is evaluated by comparing the measurement date of the health data 40 with the reliability degree calculation date, and is rated higher as a period to the reliability degree calculation date becomes shorter. For example, in a case of evaluating in three stages, a period of three months or less is set to 3, a period of three months to one year is set to 2, and a period of one year or more is set to 1. In a case where the measurement date and time is stored in the storage memory 23 or the like, it is preferable that the reliability degree of the measurement date and time is automatically updated.

The case tendency determination unit 22 determines a case tendency value indicating a tendency of a specific case in terms of the likelihood or susceptibility to contracting a disease such as a heart disease, a stroke, diabetes (hyperglycemia), or a tumor, from a plurality of examination values included in the acquired health data 40. For example, the determination is made based on a case pattern in which the susceptibility to contracting the stroke is stored in advance using the examination values of the blood glucose level and the blood pressure. It is preferable that the case pattern is included in a case determination table (not shown) or the like in the case tendency determination unit 22. The health data 40 having the determined case tendency value is controlled to be extracted in a case of corresponding to the intended-use information, thereby controlling the output.

The case tendency is calculated as, for example, a numerical value from 0 to 99. The value is calculated as a tendency value closer to 0 as the number of signs of morbidity is small and the likelihood of contracting the disease is low, and closer to 99 as the likelihood that the subject already has the disease is high. In addition, the case tendency may be evaluated in four stages of “large”, “medium”, “small”, and “none”. Note that, in a case where the numerical value of the case tendency is less than 25 or “none”, it is preferable to determine that there is no case tendency.

The storage memory 23 stores the health data 40 acquired from the data acquisition unit 20. It is preferable that the stored health data 40 is associated with the reliability degree data 43 and the case tendency data 44. A built-in solid state drive (SSD) is preferably used for the storage. A recording medium, such as a universal serial bus (USB) memory or a hard disc drive (HDD), may be used instead of the SSD.

The output destination information acquisition unit 25 acquires output destination information having affiliation information, intended-use information, a required reliability degree, and an output condition rule of the health data utilization device 12 in a case where communication is performed with the health data utilization device 12 or in a case where the output destination of the health data 40 is registered in advance.

The intended-use information is information on the intended use of the health data 40 in the health data utilization device 12, and includes “subject recruitment” for recruiting subjects for clinical trials, “drug discovery” using results of clinical trials, “statistics” used for investigating the health status of a specific population, “insurance plan proposal” for providing a plan such as life insurance according to the health status, and “medical inquiry” and “diagnosis” for sharing information on a specific patient in regional medical collaboration. In addition, it is preferable that the intended-use information also includes information on the organization (corporation) to which the health data utilization device 12 belongs and the affiliation of the person in charge of operations.

The required case is information for designating the name of the disease or the type of the examination result included in the health data 40 in a case of requesting the health data 40 related to a specific case. For example, the health data 40 having a case tendency value or a medical image equal to or greater than a certain value for a specific disease is requested.

The required reliability degree is a lower limit value of the reliability degree required for the health data 40 in order for the health data utilization device 12, which is the output destination, to obtain the health data 40 with the required accuracy. In a case where highly accurate and precise health data 40 is required, the required reliability degree is set high, and, in a case where the accuracy and precision are not a concern, the required reliability degree is set low. The required reliability degree is in a format that can be compared with the reliability degree of the health data 40 calculated and corrected by the health data management device 11.

The output condition rule is a rule by which an institution (corporation) that owns the health data utilization device 12 sets a fee to be paid as consideration for the health data, the rule being implemented in accordance with a contract or agreement with an institution (corporation) that owns the health data management device 11, and that is applied in a case where the health data utilization device 12 provides products based on the health data 40. For example, there are dynamic pricing that allows a change in a fee according to the reliability degree comparison result, and an insurance provision condition that changes an insurance premium rate according to the reliability degree comparison result.

The health data extraction unit 26 extracts health data 40 having data corresponding to the acquired output destination information from the data acquisition unit 20 or the storage memory 23. For example, health data 40 having examination results and measurement condition items that match the intended use is selected and extracted. In the extraction, health data that matches the output range determined based on the acquired output condition, particularly the affiliation information and the intended-use information, is transmitted to the reliability degree correction unit 27 as health data that can be output. That is, the output range of the health data is determined for each measurement condition from the affiliation information and the intended-use information, and the health data is output according to the output range.

The reliability degree correction unit 27 performs weighting on each item included in the measurement conditions according to the affiliation information and the intended-use information, which are the output destination information, and corrects the reliability degree in accordance with the measurement conditions for which the weighting is performed according to an extent of the weighting. In the correction of the reliability degree, each item of the measurement conditions is corrected, and the overall reliability degree is also corrected according to the correction of each item. It is preferable that the weighting of the correction is set in advance for each combination of the affiliation information and the intended-use information, and that a weighting pattern stored in the reliability degree correction unit 27 is used.

The weighting indicates the priority of each item of the measurement conditions for the intended use or the like of the output destination, and the degree of weighting for each item is set according to the content emphasized by the intended use. For example, in a case where the precision of the examination result is important, such as in “drug discovery” or “diagnosis”, the reliability degree of the health data 40 is greatly affected by whether the measurer is qualified or not. Therefore, in a case where the measurer is a qualified person who has a specific qualification such as a doctor or a nurse, weighting is performed to a greater extent than usual.

The output data collation unit 28 has a function of providing determination logic for collating the health data 40 that meets the requirements of the output destination and determining the output condition. The requirements of the output destination are information included in the required case, the required reliability degree, and the output condition in addition to the intended-use information. Collation is performed to identify the health data 40 that meets the requirements of the output destination from the reliability degree-corrected health data 40 received from the reliability degree correction unit 27. The collation uses the required reliability degree, which is the output destination information, and the output condition.

The reliability degree comparison unit 29 compares the required reliability degree with the reliability degree of the health data 40 for each measurement condition, and acquires a comparison result. The health data 40 that satisfies the required reliability degree is specified based on the comparison result. Even in a case where there is no health data 40 that satisfies the required reliability degree, the health data 40 having a reliability degree closest to the required reliability degree is specified. The output condition determination unit 30 determines an output condition in a case of outputting the health data 40 according to the transmitted comparison result. In a case where the rule of the output condition is a content that does not affect the reliability degree, the health data 40 having a reliability degree that does not satisfy the required reliability degree is not output.

The output condition determination unit 30 can change the output condition based on the output condition rule by using the reliability degree comparison result. In the reliability degree comparison result, in a case where the reliability degree satisfies the required reliability degree, the health data 40 is output to the health data utilization device 12 without changing the output condition, and, in a case where the reliability degree does not satisfy the required reliability degree, the health data 40 is output to the health data utilization device 12 by changing the output condition. The output condition to be changed in a case where the required reliability degree is not satisfied is advantageous for an institution (corporation) that owns the health data utilization device 12.

The output control unit 31 outputs the health data 40 serving as an output target to the health data utilization device 12. The reliability degree of the health data 40 may be output to the health data utilization device 12 together with the health data 40 and notified of.

In addition, the output control unit 31 stores a program related to processing such as image processing in a program memory (not shown), and displays each data included in the health data 40 on a display as necessary.

In a case where the reliability degree data 43 and the case tendency data 44 are added to the health data 40 acquired from the medical device 13 in a state in which the output destination information acquisition unit 25 acquires the output destination information, the health data extraction unit 26 extracts the health data 40 serving as an extraction target and transmits the health data 40 to the reliability degree correction unit 27. It is preferable that the health data 40 that is not an extraction target is stored in the storage memory 23.

On the other hand, in a case where the reliability degree data 43 and the case tendency data 44 are added to the health data 40 in a state in which the output destination information acquisition unit 25 does not acquire the output destination information, the health data 40 is temporarily stored in the storage memory 23, and the health data extraction unit 26 extracts the health data 40 corresponding to the extraction target from the storage memory 23 according to the acquisition of the output destination information.

An example in which the output of the health data 40 is controlled from the output destination information in the present embodiment will be described. As a first example, a case will be described in which, as the output destination information, the intended-use information is “subject recruitment: diabetes”, the required reliability degree is “medium”, and the output condition rule is “dynamic pricing”.

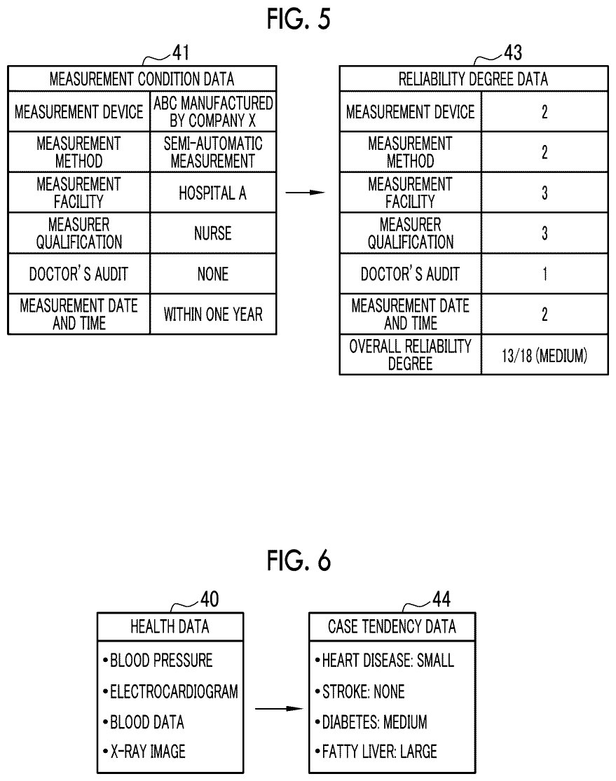

As shown in FIG. 5, reliability degree data 43 in which the reliability degree of each item of the measurement conditions and the overall reliability degree are calculated by the reliability degree calculation unit 21 is generated from the measurement condition data 41. It is preferable that the overall reliability degree is ranked based on a ratio of a total value of scores of the calculated reliability degrees of the respective items of the measurement conditions to a maximum value of a possible score. For the ranking, for example, in a case where the ratio of the total value to the maximum value is less than 50%, the ranking is assigned as “low”, in a case where the ratio of the total value to the maximum value is 50% or more and less than 75%, the ranking is assigned as “medium”, and, in a case where the ratio of the total value to the maximum value is 75% or more, the ranking is assigned as “high”.

In the measurement condition data 41, for example, in a case where the measurement device is “ABC manufactured by Company X”, the measurement method is “semi-automatic measurement”, the measurement facility is “hospital A”, the measurer qualification is “nurse”, the doctor's audit is “none”, and the measurement date and time is “within one year”, the reliability degree score is calculated using the reliability degree calculation table as “2” for the measurement device, “2” for the measurement method, “3” for the measurement facility, “3” for the measurer qualification, “1” for the doctor's audit, and “2” for the measurement date and time. This is 13 out of the maximum value of the possible score of 18, so that the ratio of the total value to the maximum value of the score is about 72%, and the rank of the overall reliability degree is “medium”.

As shown in FIG. 6, the case tendency data 44 in which the tendency of the specific case is determined by using the plurality of examination values included in the health data 40 by the case tendency determination unit 22 is generated. For example, from the health data 40 having the examination results of the blood pressure, the electrocardiogram, the blood data, and the X-ray examination image, it is determined whether the examination result such as each examination value is a normal value or an abnormal value, and the case tendency data 44 indicating the likelihood of contracting a specific disease is generated. The case tendency data 44 may be a case tendency value evaluated by a numerical value, or may be evaluated on a four-stage scale of “none”, “low”, “medium”, and “high” according to the likelihood of contracting the disease.

The generated reliability degree data 43 and case tendency data 44 are associated with the health data 40, and the health data extraction unit 26 extracts health data 40 included in the output range from the health data 40 based on the output destination information. For the setting of the output range, each item of the measurement conditions and the case tendency value is used.

In a case where the intended use is “subject recruitment” in clinical trials and the like, in order to gather subjects with the required case tendency, the health data extraction unit 26 determines whether the health data 40 is included in the output range and extracts the health data 40. In a case where the required case is “diabetes”, the health data 40 having a case tendency of diabetes in the case tendency data 44 is set as the output range.

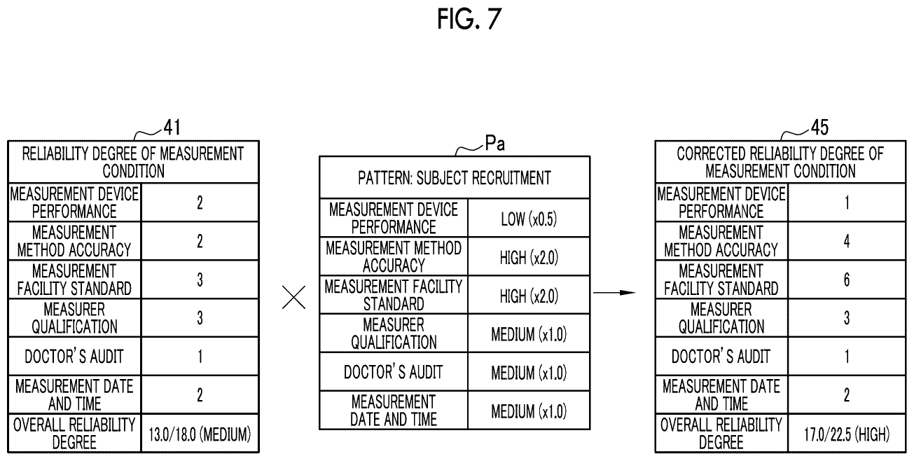

As shown in FIG. 7, the weighting pattern determined according to the intended-use information performs weighting on each item of the reliability degree data 43. In a weighting pattern Pa in a case where the intended use is “subject recruitment”, the rank (performance) of the measurement device required for the acquisition of the individual precise numerical data is of low importance, and thus the weighting is “low”, but a tendency of a data group required for determining a case is of high importance, and thus the measurement method and the measurement institution, which are information related to the measurement operation, are “high”. It is preferable that the items which are not particularly designated are “medium”. The reliability degree weighting is performed on each reliability degree data in three stages depending on the importance: “high” where the reliability degree value is doubled; “medium” where the reliability degree value is kept unchanged; and “low” where the reliability degree value is multiplied by 0.5.

In a case where the calculated reliability degree data (see FIG. 4) is corrected using the weighting pattern Pa of the intended use of “subject recruitment”, the weight for the reliability degree of “2” for the measurement device performance is corrected by applying a factor of 0.5, which corresponds to “low”, resulting in a corrected reliability degree of “1”. The weight for the reliability degree of “2” for the measurement method accuracy is corrected by applying a factor of 2, which corresponds to “high”, resulting in a corrected reliability degree of “4”, and the weight for the reliability degree of “3” for the measurement facility standard is corrected by applying a factor of 2, which corresponds to “high”, resulting in a corrected reliability degree of “6”. Since the weight for the measurer qualification, the doctor's audit, and the measurement date and time is kept unchanged, which corresponds to “medium”, the reliability degree before correction is treated as the corrected reliability degree as it is. The overall reliability degree is also updated from a maximum value of the reliability degree that can be taken according to the correction of the reliability degree of each item and a total value of the corrected reliability degrees. The overall reliability degree after the correction is 17.0 out of the maximum value of the possible score of 22.5 so that the ratio of the total value to the maximum value of the score is about 76%, and the rank of the overall reliability degree is “high”.

In the health data 40 in which the reliability degree is corrected, the output data collation unit 28 compares the required reliability degree and determines the output condition. In a case where the required reliability degree is “medium”, a comparison result is obtained in which the health data 40 having the reliability degree of “high” or “medium” satisfies the required reliability degree, and the health data 40 having the reliability degree of “low” does not satisfy the required reliability degree.

In a case where the output condition rule is a dynamic pricing output condition, the output condition is a fee paid by the output destination for outputting the health data 40, and, in a case where the reliability degree does not satisfy the required reliability degree, the fee is changed to a lower price than a normal fee. Since matching is performed with the corrected reliability degree, it is possible to set the health data 40 having a sufficient reliability degree for the request of the user as an output target. In addition, even in a case where the reliability degree of the health data 40 is insufficient, it is possible to output the health data 40 in a case where the output condition is met, for example, in a case where the acquisition cost is low.

After the output condition has been set, the health data 40 serving as an output target and its reliability degree are output to the health data utilization device 12 via the output control unit 31. The health data utilization device 12 stores the acquired health data 40 and uses the health data 40 for any intended use. It is preferable to, in a case of outputting a health data group consisting of the plurality of health data 40, set and output a minimum value of the reliability degrees provided for the plurality of health data 40 as a reliability degree of the health data group. By notifying of the health data group and the reliability degree of each health data 40, the health data utilization device 12 can easily check particularly useful health data 40 from among the plurality of acquired health data 40.

As a second example, a case will be described in which, as the output destination information, the intended-use information is “insurance plan proposal”, the required reliability degree is “high”, and the output condition rule is “insurance provision condition”. Since the health data 40 is the same as that in the first example, the description of the content common to the first example will be omitted.

In a case where the intended-use information is “insurance plan proposal”, it is preferable to extract the health data 40 in which the measurement date and time in the measurement conditions of the health data is “within one year” as the output range in order to acquire the health data 40 such as the latest health checkup result, and to transmit the health data 40 to the reliability degree correction unit 27.

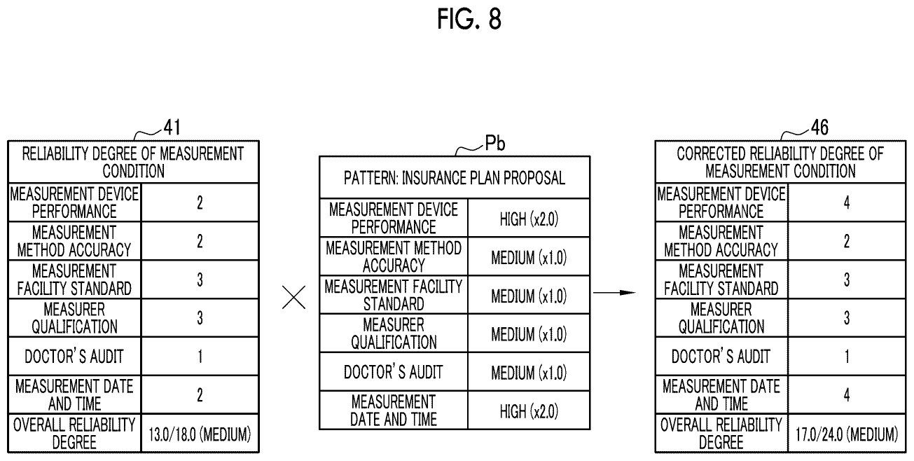

As shown in FIG. 8, since the health data 40 is preferable as it is closer to the current health status of the examinee, it is preferable that a weighting pattern Pb of the “insurance plan proposal” is corrected by setting the weight for the “measurement device performance” and the “measurement date and time” to be large. As a result of the correction, corrected reliability degree data 46 in which the overall reliability degree is “medium” is obtained.

The health data utilization device 12 proposes the created insurance plan to the medical examinee via the health data management device 11, and thus feeds back the utilization result of the acquired health data 40 to the health data management device 11. In the creation and proposal of the insurance plan, the more reliable the examination value of the health data 40 is, the smaller the risk for the insurance company is. Therefore, the output condition rule is an insurance premium rate of the insurance plan created in the health data utilization device 12, which varies depending on the reliability degree.

The output condition determination unit 30 reduces the insurance premium rate of the insurance provided to the individual in a case where the reliability degree of the health data 40 indicating the individual's health status satisfies the required reliability degree based on the reliability degree comparison result and is secured at a certain level or higher. In a case where the reliability degree of the health data 40 does not satisfy the required reliability degree and the reliability degree is not secured, the risk for the insurance company is added, and the insurance premium rate of the insurance to be provided is increased.

The health data 40 output to the health data utilization device 12 is used to create an insurance plan for each examinee based on the insurance premium rate determined as the output condition, and the insurance plan is proposed to the examinee via the health data management device 11.

As a third example, a case will be described in which, as the output destination information, the intended-use information is “diagnosis” in regional medical collaboration or the like. Since the health data 40 is the same as that in the first example, the description of the content common to the first and second examples will be omitted.

It is preferable that the health data 40 used for “diagnosis” by the health data utilization device 12 is one health data 40 for the same examinee and has a high reliability degree. Therefore, in a case where the health data management device 11 acquires a plurality of health data 40 for the same examinee, the health data management device 11 outputs the health data 40 having the highest reliability degree.

Specifically, in a state in which the health data management device 11 acquires the health data 40 of the same examinee from the medical device 13a of the hospital A, the medical device 13b of the B hospital, and the medical device 13c that is a device owned by the user, the health data management device 11 outputs the health data 40 having the highest reliability degree by using each calculated reliability degree data 43. It is preferable that the output data collation unit 28 uses the corrected reliability degree to select the highest reliability degree, but the health data extraction unit 26 may use the reliability degree before the correction to make the selection.

The health data 40 to be output may be selected based on the overall reliability degree of the required examination result instead of the reliability degree of the health data 40. In a case of using the examination result of the blood glucose level in the “diagnosis”, health data 40 having reliability degree data 43 with the highest overall reliability degree of the measurement conditions for blood glucose level measurement may be selected and output from the medical devices 13a, 13b, and 13c. The measurement condition with the highest reliability degree can be discriminated as the best measurement condition.

In a case where a plurality of health data 40 for the same examinee are acquired for the intended use other than “diagnosis”, the past medical history or health status may be used for the intended use of “medical inquiry” in regional medical collaboration or the like. Therefore, even in a case where the reliability degree is low, the output range may be set to a period such as “within the past 10 years”. On the other hand, in a case where a plurality of health data 40 are acquired for the intended use other than “diagnosis” and for different examinees, for example, in a case of “subject recruitment” in the first example, it is not necessary to limit the output range to “entire range”. In a case where an upper limit is set for the number of the pieces of health data 40 that can be output within the output range, the pieces of health data 40 may be determined as the output targets up to the upper limit in descending order of the reliability degree.

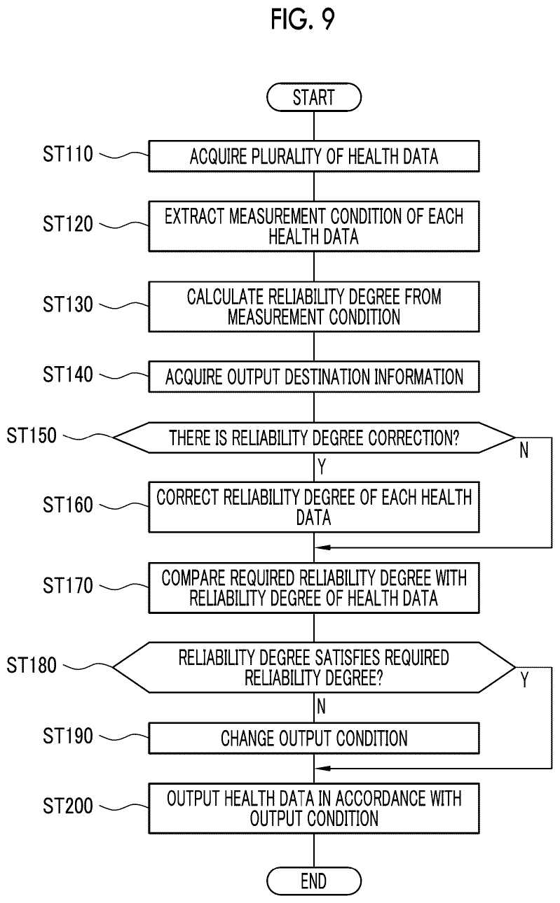

As shown in FIG. 9, a flowchart showing a series of flows until the health data management device 11 inputs the health data to the health data utilization device 12 according to the output destination information will be described. The health data management device 11 acquires a plurality of health data 40 from the medical device 13 (step ST110). The data acquisition unit 20 that acquires the health data 40 extracts the measurement conditions in the acquired health data 40 (step ST120). The reliability degree calculation unit 21 calculates the reliability degrees for the extracted measurement conditions by using the reliability degree calculation table (step ST130). The output destination information acquisition unit 25 acquires the output destination information from the health data utilization device 12 (step ST140). In a case where the weighting is performed on at least any of the items of the measurement conditions based on the output destination information (Y in step ST150), the reliability degree of each item of the measurement conditions in the health data 40 is corrected according to the determined weighting (step ST160). In a case where there is no item to be weighted in the measurement conditions (N in step ST150), the health data 40 is transmitted to the output data collation unit 28 as the reliability degree-corrected health data 40 without correcting the reliability degree of the health data 40.

The reliability degree comparison unit 29 compares the required reliability degree acquired from the output destination information with the reliability degree of the health data, and obtains a reliability degree comparison result (step ST170). In a case where the reliability degree of the health data 40 does not satisfy the required reliability degree in the reliability degree comparison result (N in step ST180), the output condition is changed (step ST190). In a case where the reliability degree of the health data 40 satisfies the required reliability degree in the reliability degree comparison result (Y in step ST180), the output condition is not changed.

The health data 40 is output to the health data utilization device 12 in accordance with the output condition determined according to the reliability comparison result (step ST200). The health data utilization device 12 that has acquired the health data 40 uses the health data 40 according to the intended use.

With the above content, by determining the processing content of the medical data based on the output destination information, it is possible to utilize the data in accordance with the laws and regulations of each country, the safety of the personal information, and the intention of the data user.

In the present embodiment, the correction of the reliability degree according to the output destination information, the comparison between the required reliability degree and the reliability degree, and the determination of the output condition are performed, but the configuration of the implementation contents may be appropriately determined according to the configuration of the output destination information. For example, in a case where the required reliability degree is the minimum value or is not set, the output condition is determined without comparing the required reliability degree with the reliability degree. In addition, in a case where the rule for determining the output condition is not based on the reliability degree of the health data 40, the output content, or the number of times of output, such as the fee incurred for outputting the health data 40 being a fixed amount for a certain period of time, the output condition is not determined, and the data to be output is collated based on whether or not the reliability degree of the health data 40 satisfies the required reliability degree.

### Second Embodiment

In the first embodiment, the health data management device 11 that determines the health data 40 to be output according to the output destination information has been described, but, in a second embodiment, the health data 40 to be output is determined based on a search performed by the user of the health data utilization device 12. Other contents that are the same as those of the first embodiment will not be described.

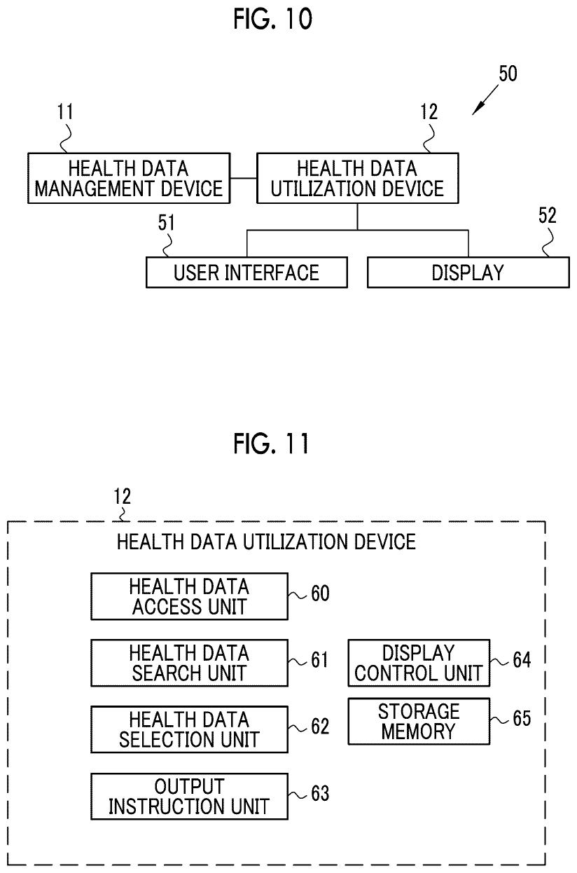

As shown in FIG. 10, in a health data management system 50 according to the second embodiment, a health data utilization device 12 that is electrically connected to a user interface (UI) 51 and a display 52 controls the output of the health data in addition to each function of the health data management device 11 (see FIG. 3) according to the first embodiment. The health data management device 11 receives a search for the health data 40 from the health data utilization device 12, which is an output destination, and outputs the health data 40 specified by the search to the health data utilization device 12.

As shown in FIG. 11, the health data utilization device 12 has functions of a health data access unit 60, a health data search unit 61, a health data selection unit 62, an output instruction unit 63, a display control unit 64, and a storage memory 65. The health data utilization device 12 has a matching logic for calculating the corrected reliability degree of the health data 40 stored in the health data management device 11 by the search and specifying the health data 40 that matches the search condition.

The health data access unit 60 accesses the plurality of health data 40 stored in the health data management device 11 and displays the health data 40 on the display 52 via the display control unit 64. The health data access unit 60 transmits information such as passwords required for access. It is preferable that the accessed health data 40 includes the reliability degree data 43 and the case tendency data 44.

The health data search unit 61 receives a user operation or the like via the user interface 51 to set search conditions for the intended use, such as a case tendency value, a measurement condition, and a reliability degree for searching for the required health data 40. In addition, a search result according to the search condition is obtained from the output data collation unit 28 of the health data management device 11 via the health data access unit 60. The search result is displayed on the display 52 via the display control unit 64.

The health data selection unit 62 selects the health data 40 to be output from the health data management device 11 to the health data utilization device 12. It is preferable that the selection of the health data 40 is performed by the user performing a cursor operation or the like on the search result displayed on the display 52.

The output instruction unit 63 issues an output instruction to output the health data 40 from the health data management device 11 based on the selected health data 40 and the output condition. The acquired health data 40 is stored in the storage memory 65.

The display control unit 64 displays a search screen in the health data selection unit 62 in a case of searching for the health data 40 stored in the health data management device 11, or displays the health data 40 acquired by being output from the health data management device 11 on the display 52.

The storage memory 65 stores the health data 40 output from the health data management device 11 and uses the health data 40 for any intended use of the health data utilization device 12 as necessary. In addition, the search condition that has been searched in the past by the health data search unit 61 is stored, and the user can call the search condition at any time.

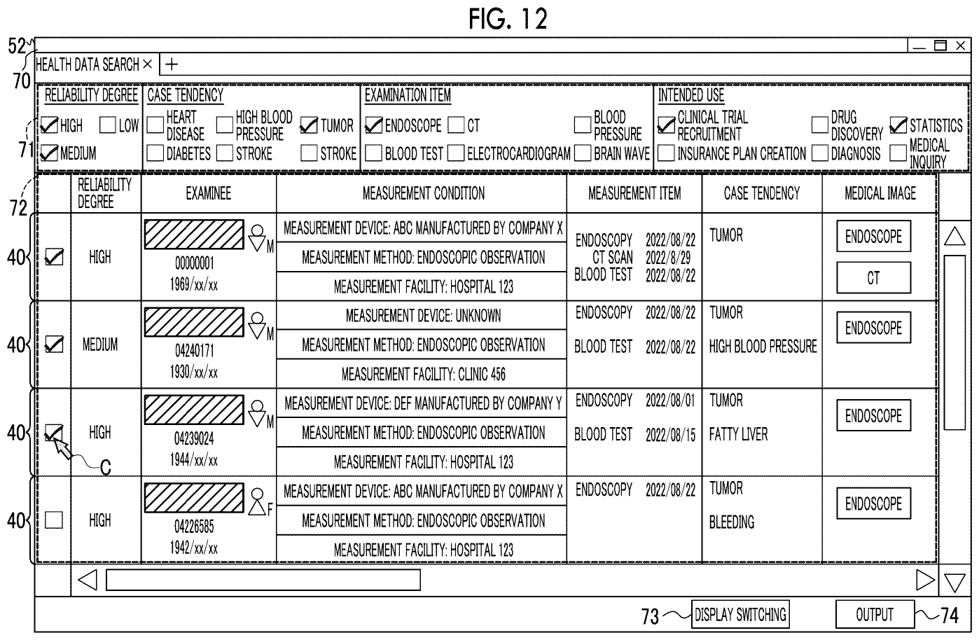

As shown in FIG. 12, the user browses the list of the health data 40 displayed on a search screen 70 displayed on the display 52 and searches for the health data 40 serving as an output target. The search screen 70 includes a search condition display region 71, a search result display region 72, a display switching button 73, and an output button 74, which are used to perform the search, selection, and output of the health data 40 stored in the health data management device 11.

In the search condition display region 71, the reliability degree of the health data 40, case tendency, examination items, and items desired by the user for the intended use of the health data 40 are added to the search conditions. Each item is added to the search condition item by selecting a check box of each item with a cursor C via the user interface 51. It is preferable that the item added to the search condition is removed from the search conditions by selecting the check box again.

In selecting the intended use, it is possible to switch between a correction search mode in which the reliability degree correction in the first embodiment is performed and a non-correction search mode in which no correction is performed. In the correction search mode, by setting the number of types of the intended use that can be selected for one search to one, the search result of the health data 40 having the corrected reliability degree based on the intended use can be obtained. By correcting the reliability degree, it is possible to output the health data 40 that is particularly suitable for the intended use in addition to the specific setting of the search condition by the user.

In the non-correction search mode, since the reliability degree correction based on the intended use is not performed, a plurality of intended uses can be selected at once. By performing the search in a state in which the reliability degree correction is not performed, the pieces of health data 40 that match the plurality of intended uses can be output.

In the search result display region 72, the pieces of health data 40 that match the search conditions selected in the search condition display region 71 are displayed in a list. The health data 40 may be displayed each time an item is selected, or a search execution button (not shown) may be provided and the search execution button may be selected with the cursor C after the search conditions are selected. The pieces of health data 40 displayed in a list are set to an output candidate state by selecting a check box with the cursor C or the like.

In the list display, the presence or absence of the reliability degree, the examinee information, the measurement condition, the examination item, the case tendency, and the medical image in the health data 40 is displayed. Information for specifying an individual in the examinee information may be displayed after being anonymized or pseudonymized, for example, by masking the name of the examinee, according to an affiliation relationship between the health data management device 11 and the health data utilization device.

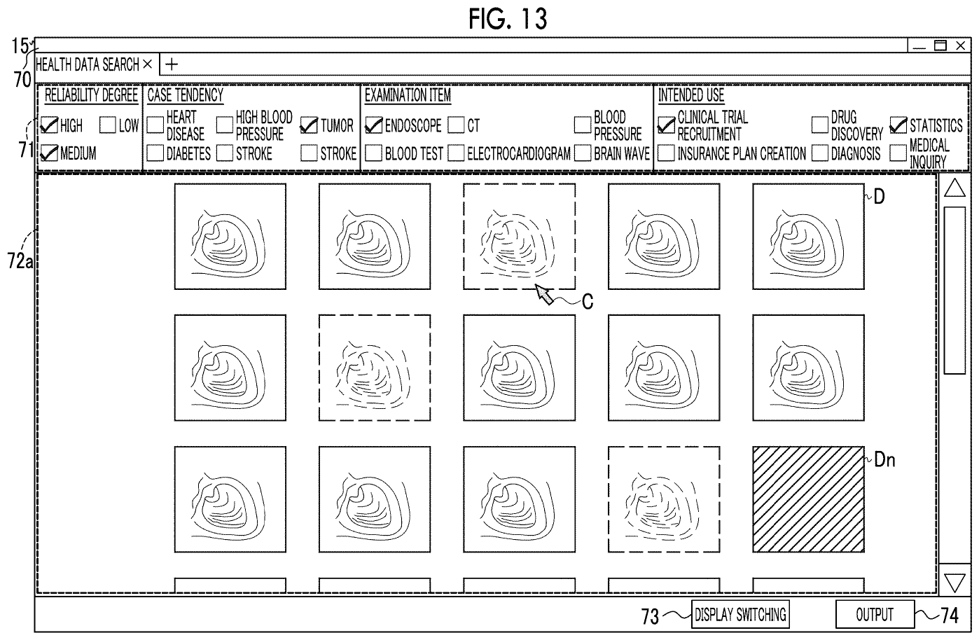

The display switching button 73 is a button that is selected in a case of switching a display aspect of the search result displayed in the search result display region 72. The selection of the display switching button 73 switches the list display (see FIG. 12), the screen list display (see FIG. 13), or the combined display (not shown) of the image and any item in the search result display region 72. The output button 74 is a button that is selected in a case of outputting the health data 40 in the output candidate state.

As shown in FIG. 13, a display aspect in a search result display region 72a may be a list display of the medical images D included in each health data 40. For example, in a case where a search is performed for “endoscope” as the examination item, the medical image D, which is an in-vivo image captured by the endoscope, is displayed. It is preferable that one or more medical images D are displayed for each health data 40 in the search result display region 72a, and it is preferable that the health data 40 that does not include or cannot display the medical image D is displayed as an icon having the same size as the medical image D, such as an image Dn. It is preferable that the medical image D is an image of a lesion or the like created in a case of diagnosis by a doctor.

It is preferable that, in a case where the medical image D or the image Dn is selected by an operation different from the user's selection of the output candidate, a pop-up window is displayed or the screen is switched so that details of the health data 40 of each image can be confirmed. It is preferable that the display aspect of the image in which the output candidate state is set by the selection of the cursor C is changed.

Since the user performs the search with the corrected reliability degree, it is possible to accurately set the health data 40 having a sufficient reliability degree for the intended use as the output target. In addition, by adding the confirmation of the health data 40 by the user in addition to the search based on the intended-use information or the reliability degree, the health data 40 can be output in a state where unnecessary health data 40 is excluded or specific each item of the health data 40 is confirmed.

In each embodiment, the hardware structures of processing units that execute various types of processing, such as the central control unit, the input reception unit, the data acquisition unit 20, the reliability degree calculation unit 21, the case tendency determination unit 22, the output destination information acquisition unit 25, the health data extraction unit 26, the reliability degree correction unit 27, the output data collation unit 28, the reliability degree comparison unit 29, the output condition determination unit 30, the output control unit 31, the health data access unit 60, the health data search unit 61, the health data selection unit 62, the output instruction unit 63, and the display control unit 64, are various processors as shown below. The various processors include a central processing unit (CPU) that is a general-purpose processor that executes software (programs) to function as various processing units, a programmable logic device (PLD) that is a processor capable of changing a circuit configuration after manufacture, such as a field programmable gate array (FPGA), and an exclusive electric circuit that is a processor having a circuit configuration exclusively designed to execute various kinds of processing.

One processing unit may be configured of one of these various processors, or may be configured of a combination of two or more processors of the same type or different types (for example, a combination of a plurality of FPGAs or a combination of a CPU and an FPGA). In addition, a plurality of processing units may be configured of one processor. As an example in which the plurality of processing units are configured of one processor, first, as typified by computers such as a client or a server, one processor is configured of a combination of one or more CPUs and software, and this processor functions as the plurality of processing units. Second, as typified by a system on chip (SoC) or the like, a processor that realizes the functions of the entire system including the plurality of processing units by using one integrated circuit (IC) chip is used. As described above, the various processing units are configured using one or more of the various processors as a hardware structure.

Furthermore, the hardware structure of the various processors is more specifically an electric circuit (circuitry) having a form in which circuit elements such as semiconductor elements are combined. In addition, a hardware structure of a storage unit is a storage device such as a hard disc drive (HDD) or a solid state drive (SSD). In addition, from the above description, it is possible to understand the health data management device, the health data management program, and the health data management method described in the following appendices 1 to 13.

### APPENDIX 1

A health data management device comprising:
a processor,
in which the processor is configured to
   acquire affiliation information of an output destination of a plurality of health data measured under different measurement conditions and intended-use information of the health data, and
   control output of the health data for each measurement condition based on the affiliation information and the intended-use information.

### APPENDIX 2

The health data management device according to Appendix 1,
in which the measurement conditions include at least one of the following items: a measurement date and time; a measurement device; a measurement method; a measurement location; and a qualification of a measurer, and
the processor is configured to
   determine a reliability degree for the health data from the measurement conditions, and
   control the output of the health data according to the reliability degree.

### APPENDIX 3

The health data management device according to Appendix 2,
in which the processor is configured to
   perform weighting on the item included in the measurement conditions from the affiliation information and the intended-use information, and
   correct the reliability degree in accordance with the measurement conditions for which the weighting is performed.

### APPENDIX 4

The health data management device according to Appendix 3,
in which the processor is configured to perform the weighting to a greater extent in a case where the measurer is a qualified person who has a specific qualification than in a case where the measurer is an unqualified person.

### APPENDIX 5

The health data management device according to Appendix 3 or 4,
in which the output destination sets a required reliability degree that is a lower limit value of the reliability degree of the health data whose output is received, and
the processor is configured to
   compare the required reliability degree with the reliability degree of the health data for each measurement condition, and
   determine an output condition for outputting the health data according to a comparison result.

### APPENDIX 6

The health data management device according to any one of Appendices 3 to 5,
in which the processor is configured to
   output the health data without changing the output condition in a case where the reliability degree satisfies the required reliability degree, and
   output the health data by changing the output condition in a case where the reliability degree does not satisfy the required reliability degree.

### APPENDIX 7

The health data management device according to Appendix 6,
in which the output condition is a fee paid by the output destination to an output source for the output of the health data, and
in a case where the reliability degree does not satisfy the required reliability degree, the fee is changed to a lower price than a normal fee.

### APPENDIX 8

The health data management device according to any one of Appendices 2 to 7,
in which the processor is configured to, in a case where there are the plurality of health data for the same examinee, output the health data having a highest reliability degree.

### APPENDIX 9

The health data management device according to any one of Appendices 2 to 8,
in which the processor is configured to notify the output destination of the reliability degree of the health data to be output.

### APPENDIX 10

The health data management device according to Appendix 9,
in which the processor is configured to, in a case of outputting a health data group consisting of the plurality of health data, set a minimum value of the reliability degrees provided for the plurality of health data as a reliability degree of the health data group.

### APPENDIX 11

The health data management device according to any one of Appendices 1 to 10,
in which the processor is configured to
   determine an output range of the health data for each measurement condition based on the affiliation information and the intended-use information, and
   output the health data according to the output range.

### APPENDIX 12

The health data management device according to any one of Appendices 1 to 11,
in which the processor is configured to
   determine a case tendency value, which is a value indicating a tendency of a specific case, from a plurality of examination values included in the health data, and
   in a case where the intended-use information includes information on a required case tendency value, control the output of the health data based on the case tendency value corresponding to the intended-use information.

### APPENDIX 13

The health data management device according to any one of Appendices 1 to 12,
in which the processor is configured to
   receive a search for the health data from the output destination, and
   output the health data specified by the search.

### EXPLANATION OF REFERENCES

10: health data management system
11: health data management device
12: health data utilization device
13: medical device
13a: medical device
13b: medical device
13c: medical device
20: data acquisition unit
21: reliability degree calculation unit
22: case tendency determination unit
23: storage memory
25: output destination information acquisition unit
26: health data extraction unit
27: reliability degree correction unit
28: output data collation unit
29: reliability degree comparison unit
30: output condition determination unit
31: output control unit
40: health data
41: measurement condition data
43: reliability degree data
44: case tendency data
45: corrected reliability degree data
46: corrected reliability degree data
50: health data management system
51: user interface
52: display
60: health data access unit
61: health data search unit
62: health data selection unit
63: output instruction unit
64: display control unit
65: storage memory
70: search screen
71: search condition display region
72: search result display region
72a: search result display region
73: display switching button
74: output button
C: cursor
D: medical image
Dn: image
Pa: weighting pattern
Pb: weighting pattern
ST110 to ST200: step

What is claimed is:

1. A health data management device comprising:
a processor,
wherein the processor is configured to
acquire affiliation information of an output destination of a plurality of health data measured under different measurement conditions and intended-use information of the health data, and
control output of the health data for each measurement condition based on the affiliation information and the intended-use information.

2. The health data management device according to claim 1,
wherein the measurement conditions include at least one of the following items: a measurement date and time; a measurement device; a measurement method; a measurement location; and a qualification of a measurer, and
the processor is configured to
determine a reliability degree for the health data from the measurement conditions, and
control the output of the health data according to the reliability degree.

3. The health data management device according to claim 2,
wherein the processor is configured to
perform weighting on the item included in the measurement conditions based on the affiliation information and the intended-use information, and
correct the reliability degree in accordance with the weighted measurement conditions.

4. The health data management device according to claim 3,
wherein the processor is configured to perform the weighting to a greater extent in a case where the measurer is a person with a specific qualification than in a case where the measurer is unqualified.

5. The health data management device according to claim 3,
wherein the output destination sets a required reliability degree, which is a lower limit of the reliability degree acceptable for the health data to be output, and
the processor is configured to
compare the required reliability degree with the reliability degree of the health data for each measurement condition, and
determine an output condition for outputting the health data according to a comparison result.

6. The health data management device according to claim 5,
wherein the processor is configured to
output the health data without changing the output condition in a case where the reliability degree satisfies the required reliability degree, and
output the health data by changing the output condition in a case where the reliability degree does not satisfy the required reliability degree.

7. The health data management device according to claim 6,
wherein the output condition is a fee paid by the output destination to an output source for the output of the health data, and
in a case where the reliability degree does not satisfy the required reliability degree, the fee is changed to a lower price than a normal fee.

8. The health data management device according to claim 2,
wherein the processor is configured to, in a case where there are a plurality of health data for the same examinee, output the health data having the highest reliability degree.

9. The health data management device according to claim 2,
wherein the processor is configured to notify the output destination of the reliability degree of the health data to be output.

10. The health data management device according to claim 9,
wherein the processor is configured to, in a case of outputting a health data group consisting of a plurality of health data, set the lowest reliability degree among those of the plurality of health data as the reliability degree of the health data group.

11. The health data management device according to claim 1,
wherein the processor is configured to
determine an output range of the health data for each measurement condition based on the affiliation information and the intended-use information, and
output the health data according to the output range.

12. The health data management device according to claim 1,
wherein the processor is configured to
determine a case tendency value, which is a value indicating a tendency of a specific case, from a plurality of examination values included in the health data, and
in a case where the intended-use information includes information on a required case tendency value, control the output of the health data based on the case tendency value corresponding to the intended-use information.

13. The health data management device according to claim 1,
wherein the processor is configured to
receive a search for the health data from the output destination, and
output the health data specified by the search.

14. A non-transitory computer readable medium for storing a health data management program, wherein the health data management program causing a computer to execute:
a function of acquiring affiliation information of an output destination of a plurality of health data measured under different measurement conditions and intended-use information of the health data; and
a function of controlling output of the health data for each measurement condition based on the affiliation information and the intended-use information.

15. A health data management method comprising:
a step of acquiring affiliation information of an output destination of a plurality of health data measured under different measurement conditions and intended-use information of the health data; and
a step of controlling output of the health data for each measurement condition based on the affiliation information and the intended-use information.
